(19) United States
(12) Patent Application Publication
(10) Pub. No.: US 2020/0384040 A1
(43) Pub. Date: Dec. 10, 2020

(54) USE OF BACTEROIDES FRAGILIS IN PREPARATION OF MEDICAMENT FOR TREATING AND PREVENTING TUMOR

(71) Applicant: SHENZHEN YUEYAO LIFE TECHNOLOGY CO., LTD., Shenzhen, Guangdong (CN)

(72) Inventor: Gucheng ZENG, Guangzhou City (CN)

(21) Appl. No.: 16/648,774

(22) PCT Filed: Sep. 28, 2017
(86) PCT No.: PCT/CN2017/103940

§ 371 Date: Jul. 6, 2020

(30) Foreign Application Priority Data
Sep. 22, 2017 (CN) 201710865410.6

(51) Int. Cl. A61K 35/741; A23L 33/135; A61P 35/00

(52) U.S. Cl. CPC A61K 35/741; A61P 35/00; A23L 33/135

(57) ABSTRACT

Provided are uses of Bacteroides fragilis and a therapeutic or prophylactic composition containing Bacteroides fragilis in the treatment and/or prevention of tumors. Also provided are uses of Bacteroides fragilis and a composition containing Bacteroides fragilis in the preparation of a medicament for the treatment and/or prevention of tumors, which can significantly inhibit the growth of tumors. Also provided are uses of Bacteroides fragilis in the preparation of pharmaceutical compositions, foodstuffs, health products and food additives for the treatment and/or prevention of tumors.

### CROSS REFERENCE TO RELATED APPLICATIONS

This application claims priority to PCT Application No. PCT/CN2017/103940, having a filing date of Sep. 28, 2017, which is based on Chinese Application No. 201710865410.6, having a filing date of Sep. 22, 2017, the entire contents both of which are incorporated herein by reference.

### FIELD OF TECHNOLOGY

The following relates to the technical field of biomedicine, particularly, it relates to the uses of Bacteroides fragilis or a composition comprising Bacteroides fragilis to treat and/or prevent tumors.

### BACKGROUND

Cancer has become the “first killer” of human beings. The Global Cancer Report 2014, published by the World Health Organization (WHO), is supervised by the International Cancer Research Centre (IARC) which is the official cancer institute of WHO, and it is compiled by more than 250 scientists from more than 40 countries. The report comprehensively describes and analyzes overall situation and epidemic trends of 28 types of cancers in more than 180 countries. The report also predicts that global cancer cases will increase rapidly, and will increase year by year from 14 million in 2012 to 19 million in 2025, and to 24 million in 2035. The main difficulty in cancer treatment, especially in the treatment of advanced tumors in intermediate and advanced stage, is the inability to control cancer metastasis. Chemotherapy is currently recognized as the main treatment method, and the reason is to kill cancer cells in the body. But chemotherapeutic drugs also damage normal human cells while killing cancer cells. Moreover, chemotherapeutic drugs, especially being administered in large doses, can induce genomic disorder, increase the formation of heterogeneity and outlier and promote drug resistance, thus more aggressive cells are formed, causing cancer to further spread. Clinical results show that chemotherapy is effective for only about 7% of cancers actually. In recent years, more and more attention has been paid on immunotherapy of cancer. In the Annual Report of the ASCO Cancer Research Progress in 2016 published by the American Society of Clinical Oncology (ASCO) on Feb. 4, 2016, immunotherapy was named the biggest progress in cancer research in 2015. Just as Dr. Julie M. Vose, the chairman of ASCO, said, “Immunotherapy is the most revolutionary breakthrough in the cancer field, and this new therapy not only can improve the lives of patients, but also give the direction for future research.” Current tumor immunotherapy will become the fourth major cancer treatment after surgery, radiotherapy and chemotherapy. Therefore, it has become a worldwide research hotspot to develop safe, low-priced, highly effective and low side-effect cancer immune drugs.

The uses of bacteria in cancer treatment can be dated back to the late nineteenth century, and there are even earlier reports on bacteria's effectiveness in treating cancer. Probiotics are a type of active microorganism that are beneficial to the host, and they are active beneficial microorganism colonizing the human intestinal tract and reproductive system and producing exact health effects to improve the host's micro-ecological balance and exert beneficial effects. The probiotics are capable of relieving lactose intolerance, adjusting intestinal micro-ecology and enhancing autoimmunity, etc. After probiotics are ingested by humans and animals, they can settle on the intestinal mucosa and build colonies in the intestine to prevent harmful microorganisms from adhering thereon. They are helpful to keep humans and animals healthy by maintaining the natural microflora in the intestine and by promoting the formation of healthy and grow-able microbial preparations in individual organisms. More and more researchers are now focusing on probiotics and are gradually realizing their good therapeutic effects. Much of the recent work on bacterial therapies for cancer has focused on non-pathogenic strains. Bifidobacterium is a non-pathogenic obligate anaerobic bacterium, and has been successfully used for tumor targeting and as a therapeutic carrier, but without oncolysis. In recent years, Escherichia coli and pneumobacillus are used for cancer tumors of intestine and lung respectively in some researches, and they act as “site-specific immunomodulators” and play a more significant role in inhibiting tumor growth.

Bacteroides fragilis is an obligate anaerobic bacterium which is Gram-negative, rod-shaped, and non-motile, having obtuse and hyperchromatic ends as well as a capsule, without spores. The Bacteroides fragilis can be classified into an enterotoxigenic type and a non-enterotoxigenic type. As a part of the normal flora of human and animal intestines, Bacteroides fragilis mainly exists in the colon, and besides, it can also colonize and grow in the respiratory tract, the gastrointestinal tract and the urogenital tract. Numerous researches have shown that Bacteroides fragilis has a good effect on the prevention and treatment of acute and chronic enteritis, dysbacteriosis, upper respiratory infection and neurosis, etc.

### SUMMARY

An aspect relates to a method capable of treating and/or preventing tumors, for the above-mentioned defects that currently exist in the treatment of tumors.

It is one aspect to provide a use of Bacteroides fragilis in the treatment and/or prevention of tumors.

The tumors described in the present disclosure may be various solid tumors, such as but not limited to murine melanoma B16-F10, human liver cancer BEL7405 and SMMC7721, human prostate cancer PC-3, human breast cancer MCF-7, human fibrosarcoma cells HT1080, murine bladder sarcoma cells MB-49 and murine glioma C6 and other solid tumors.

In some embodiments, the method of treating and/or preventing a tumor is applied in combination with other methods of treating and/or preventing a tumor. In some embodiments, the other methods of treating and/or preventing tumors include, but are not limited to, chemotherapy, radiation therapy, gene therapy, surgery, or a combination thereof. Also described herein are therapeutic and prophylactic compositions comprising Bacteroides fragilis. In some embodiments, the therapeutic and prophylactic composition contains Bacteroides fragilis. In some embodiments, the therapeutic and prophylactic composition does not contain other microbial strains. In one aspect, the Bacteroides fragilis can delay the tumor growth. In one aspect, the tumor is a solid tumor. In some embodiments, the tumor includes, but is not limited to melanoma.

According to one aspect of the present disclosure, the Bacteroides fragilis in the above-mentioned use is any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

It is another aspect to provide a use of Bacteroides fragilis in the preparation of a medicament for treating and/or preventing a tumor, wherein the tumor is melanoma, or a carcinomaof the breast, liver, lung, skin, oral cavity, esophagus, stomach, intestinal tract, kidney, prostate, brain, nervous system, bladder, lymph or pancreas and so on.

It is another aspect of the present disclosure to provide a pharmaceutical composition for treating and/or preventing a tumor, wherein the pharmaceutical composition includes a pharmaceutically effective dose of Bacteroides fragilis and its pharmaceutically acceptable carrier, and the Bacteroides fragilis is an active ingredient.

In the above-mentioned pharmaceutical composition, the Bacteroides fragilis is any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

In the above-mentioned pharmaceutical composition, the pharmaceutical composition may be in any one or more dosage forms that are pharmaceutically feasible, including but are not limited to tablets, capsules, oral solutions, or lyophilized powders.

In the above-mentioned pharmaceutical composition, the pharmaceutically acceptable carrier is one or a mixture of at least two of the following: skim milk, lactose, glucose, sucrose, sorbitol, mannose, trehalose, starch, Arabic gum, calcium phosphate, alginate, gelatin, calcium silicate, fine crystalline cellulose, polyvinylpyrrolidone, cellulose, water, syrup, methyl cellulose, methyl hydroxybenzoate, propyl hydroxybenzoate, talc, magnesium stearate or mineral oil.

It is another aspect to provide a foodstuff for treating and/or preventing a tumor, wherein the foodstuff includes Bacteroides

In the above-mentioned foodstuff, the Bacteroides fragilis is any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

It is another aspect to provide a food additive for treating and/or preventing a tumor, wherein the food additive includes Bacteroides

In the above-mentioned food additive, the Bacteroides fragilis is any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

It is another aspect to provide a health product for treating and/or preventing a tumor, wherein the health product includes Bacteroides

In the above-mentioned health product, the Bacteroides fragilis is any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

A murine melanoma model is created with a transplanted tumor research method in the disclosure, and the effect of Bacteroides fragilis are detected and identified in the mouse melanoma model. Through experiments, the disclosure proves that the Bacteroides fragilis can significantly inhibit melanoma from surviving in vitro and can effectively inhibit the growth of transplanted tumors in mice, showing that it is important to develop and apply Bacteroides fragilis in clinical treatment of tumors.

### BRIEF DESCRIPTION

Some of the embodiments will be described in detail, with reference to the following figures, wherein like designations denote like members, wherein:

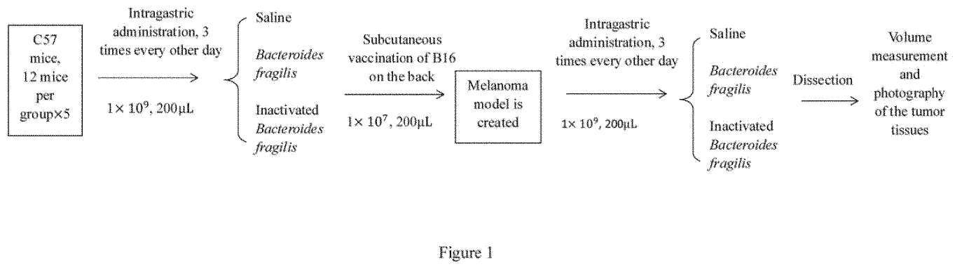

FIG. 1 is a schematic flow chart of an experiment for detecting the therapeutic effect of Bacteroides fragilis and inactivated Bacteroides fragilis on melanoma in mice;

FIG. 2 shows a detection of the inhibitory effect of Bacteroides fragilis and inactivated Bacteroides fragilis on tumorigenesis of transplanted human tumors in mice;

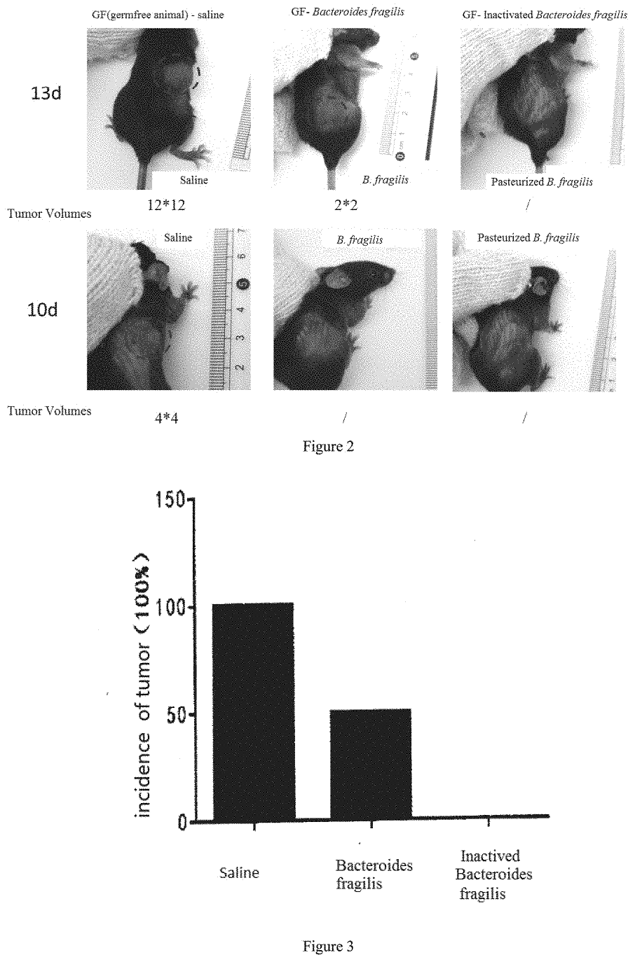

FIG. 3 shows a detection of the inhibitory effect for Bacteroides fragilis and inactivated Bacteroides fragilis on the growth of transplanted human tumors in mice; and

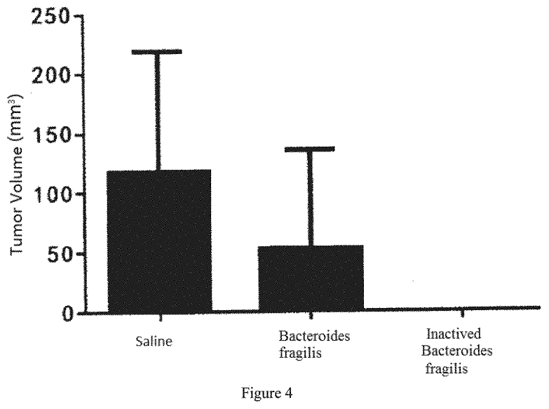

FIG. 4 is a comparison of melanoma volume in mice after treatment with Bacteroides fragilis.

### DETAILED DESCRIPTION

The present disclosure will be further described below with reference to the embodiments. It should be pointed out that after the Bacteroides fragilis for treating and/or preventing a tumor in the present disclosure, or the pharmaceutical composition, foodstuff, health product and food additive containing the Bacteroides fragilis of the present disclosure can be applied to the indications described above and exhibits the functions described above, after they are administered to the subject. The tests have been done in all dosage forms within the scope of the present disclosure. In the following, only a small part is described in the examples just for illustration, however, it should not be construed as a limitation of the disclosure.

The Bacteroides fragilis referred in the present disclosure includes, but is not limited to, any one of living Bacteroides fragilis, inactivated Bacteroides fragilis, Bacteroides fragilis lysate, and/or Bacteroides fragilis culture supernatant.

The tumor is a solid tumor. In some embodiments, the tumor includes, but is not limited to, melanoma.

The present disclosure also provides an anti-tumor pharmaceutical composition comprising a pharmaceutically effective dose of Bacteroides fragilis, wherein the so-called “pharmaceutically effective dose” is 10⁶-10¹⁰ CFU, or 10⁹ CFU. The Bacteroides fragilis includes any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant. The pharmaceutical composition is in a form including, but is not limited to, a tablet, a capsule, an oral solution, or lyophilized powders. The pharmaceutically acceptable carrier includes, but is not limited to one or more of skim milk, lactose, glucose, sucrose, sorbitol, mannose, trehalose, starch, Arabic gum, calcium phosphate, alginate, gelatin, calcium silicate, fine crystalline cellulose, polyvinylpyrrolidone, cellulose, water, syrup, methyl cellulose, methyl hydroxybenzoate, propyl hydroxybenzoate, talc, magnesium stearate, or mineral oil.

The Bacteroides fragilis of the present disclosure can also be made into foodstuffs, health products or food additives. The foodstuffs, health products or food additives all contain any one of the living Bacteroides fragilis; genetically recombined, altered or modified, attenuated, chemically treated, physically treated or inactivated Bacteroides fragilis; Bacteroides fragilis lysate; and/or Bacteroides fragilis culture supernatant. These foodstuffs, health products or food additives can all be used to treat and/or prevent tumors.

### Example 1 Bacteroides fragilis Culture

Culture Method

Step 1: A lyophilized preserved Bacteroides fragilis strain is taken, then 200 μL Tryptic Soy Broth (TSB) is added to reconstitute it. 20 uL bacterial solution is pipetted, and is streaked on the blood agar plate. Then the plate is placed in a biochemical incubator after an air exhaust processing by an anaerobic jar gassing system, and cultured in anaerobic environment at 37° C. for 48 h;

Step 2: Monoclonal colony is selected and inoculated in 10 mL TSB, followed by being cultured in anaerobic environment at 37° C. for 12 h;

Step 3: 1% (v/v) of strain is inoculated in 500 mL TSB in a flask and cultured in anaerobic environment at 37° C. for 48 hours.

Step 4: The bacterial solution is collected and centrifuged at 6000 rpm for 10 min. The bacterial sludge is washed twice with physiological saline, and is finally reconstituted with physiological saline for later use. The viable bacteria are counted.

### Example 2 Experiment of the Therapeutic Effect of Bacteroides fragilis on Tumors (Melanoma) in Mice

FIG. 1 is a schematic flow chart of an experiment for detecting the therapeutic and preventive effects of Bacteroides fragilis and inactivated Bacteroides fragilis on tumors (melanoma) in mice.

1. Culture Method

The culture method of Bacteroides fragilis is the same as that in Example 1.

2. Sample Preparation

1) Preparation of Living Bacteroides fragilis ZY-312

Step 1: A lyophilized preserved strain is taken, and then 200 μL lyophilized preserved bacterial culture medium is added to reconstitute it. Then 20 μL bacterial solution is pipetted, and is streaked on the blood agar plate. The plate is placed in a biochemical incubator after an air exhaust processing by an anaerobic jar gassing system, and then is cultured in anaerobic environment at 37° C. for 48 h;

Step 2: Monoclonal colony is selected and inoculated in 10 mL TSB, and followed by being cultured in anaerobic environment at 37° C. for 12 h;

Step 3: 1% (v/v) of strain is inoculated in 500 mLTSB in a flask, and cultured in anaerobic environment at 37° C. for 48 hours.

Step 4: The bacterial solution is taken and centrifuged with a centrifuge at 6000 rpm for 10 min. The bacterial sludge is washed twice with physiological saline, and is finally reconstituted with physiological saline for later use. The viable bacteria are counted.

2) Inactivated Bacteroides fragilis

The bacterial solution is heated in a water bath at 70° C. for 30 min to obtain inactivated bacterial solution.

3) Bacteroides fragilis Lysate

The culture bacterial solution of Bacteroides fragilis is treated with an ultrasonic crushing method by using an ultrasonic crusher, crushed for 2 seconds, stopped for 5 seconds, with a lasting time of 20 minutes, to obtain a Bacteroides fragilis lysate.

4) Bacteroides fragilis Culture Supernatant

The culture bacterial solution of Bacteroides fragilis was centrifuged with a centrifuge at 6000 rpm for 10 min to obtain a culture supernatant of Bacteroides fragilis

3. Mice Experiments of Effect of Bacteroides fragilis on Prevention and Treatment of Tumors

Experimental animals: Thirty-six C57BL/6 mice, 3 to 4 weeks old, in good mental state, are purchased from the Experimental Animal Center of Sun Yat-sen University. The mice are randomly divided into 3 groups, 12 mice for each, and the 3 groups are the control group, the living bacteria gavage group, and the inactivated bacteria gavage group, respectively.

Experimental Process and Results:

10⁹ CFU of Bacteroides fragilis, inactivated Bacteroides fragilis, and saline control are respectively administered intragastrically to the three groups of mice three times every other day for 1 week, and the body weights of the mice are measured daily. The murine tumor (melanoma) cells B16 are digested with TE after they are grown to logarithmic phase, and the medium is used for neutralizing. The cells are collected through centrifugation, and then washed twice with DPBS to remove the residual serum. The cells are resuspended with DPBS and counted. 10⁷ cells are inoculated subcutaneously on the back of each mouse, and the mice are kept being treated by gavage. After 1 week, the tumor-bearing mice are killed to obtain the subcutaneous tumors, and tumor volume is measured. The results show that both Bacteroides fragilis and the inactivated bacteria can significantly inhibit formation and growth of murine tumor (FIG. 2 and FIG. 3). In addition, the experimental results in FIG. 4 show that the murine tumors (melanoma) in the control group become larger gradually over time. While the growth rate of tumor in each Bacteroides fragilis gavage group is significantly slower, and no tumor is formed, or the tumor volume is significantly smaller than that in the saline control group, (the difference is statistically significant, p<0.01), which indicates that Bacteroides fragilis has induced an anti-tumor effect in the body and inhibited the tumor growth, having a good effect on the prevention and treatment of tumors, such as melanoma.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method for at least one of treating and preventing a tumor, comprising, administering Bacteroides fragilis to a subject in need thereof.

2. The method according to claim 1, wherein the Bacteroides fragilis is at least one of the following: living Bacteroides fragilis; genetically recombined, altered or modified, attenuated, chemically treated, physically treated or inactivated Bacteroides fragilis; Bacteroides fragilis lysate; and Bacteroides fragilis culture supernatant.

3. The method according to claim 1, wherein the tumor is selected from various solid tumors.

4. A method for treating and/or preventing a tumor, comprising administering a medicament of Bacteroides fragilis to a subject in need thereof, the tumor is melanoma; or a carcinoma of the breast, liver, lung, skin, oral cavity, esophagus, stomach, intestinal tract, kidney, prostate, brain, nervous system, bladder, lymph or pancreas.

5. A pharmaceutical composition for treating and/or preventing a tumor, wherein the pharmaceutical composition includes a pharmaceutically effective dose of Bacteroides fragilis and its pharmaceutically acceptable carrier.

6. The pharmaceutical composition according to claim 5, wherein the Bacteroides fragilis is any one of the following: living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

7. The pharmaceutical composition according to claim 5, wherein the pharmaceutical composition is in a form of tablet, capsule, oral solution, or lyophilized powders.

8. The pharmaceutical composition according to claim 5, wherein the pharmaceutically acceptable carrier is one or a mixture of at least two of the following: skim milk, lactose, glucose, sucrose, sorbitol, mannose, trehalose, starch, Arabic gum, calcium phosphate, alginate, gelatin, calcium silicate, fine crystalline cellulose, polyvinylpyrrolidone, cellulose, water, syrup, methyl cellulose, methyl hydroxybenzoate, propyl hydroxybenzoate, talc, magnesium stearate or mineral oil.

9. A foodstuff for treating and/or preventing a tumor, wherein the foodstuff includes at least one of the following: living Bacteroides fragilis; genetically recombined, altered or modified, attenuated, chemically treated, physically treated or inactivated Bacteroides fragilis; Bacteroides fragilis lysate; and Bacteroides fragilis culture supernatant.

10. A health product for treating and/or preventing a tumor, wherein the health product includes at least one of the following: living Bacteroides fragilis; genetically recombined, altered or modified, attenuated, chemically treated, physically treated or inactivated Bacteroides fragilis; Bacteroides fragilis lysate; and Bacteroides fragilis culture supernatant.

11. A food additive for at least one of treating and/or preventing a tumor, wherein the food additive includes at least one of the following: living Bacteroides fragilis; genetically recombined, altered or modified, attenuated, chemically treated, physically treated or inactivated Bacteroides fragilis; Bacteroides fragilis lysate; and Bacteroides fragilis culture supernatant.
